(19) United States
(12) Patent Application Publication
(10) Pub. No.: US 2012/0109082 A1
(43) Pub. Date: May 3, 2012

(54) DEVICES FOR DELIVERING AT LEAST ONE ACTIVE AGENT TO TISSUE

(75) Inventors: Charles Leahy, Concord, MA (US); Robert F. Thompson, Weston, MA (US); Edward Ellis, Lynnfield, MA (US)

(73) Assignee: VISTA SCIENTIFIC LLC, Andover, MA (US)

(21) Appl. No.: 13/284,892

(22) Filed: Oct. 29, 2011

Related U.S. Application Data
(60) Provisional application No. 61/408,016, filed on Oct. 29, 2010.
(60) Provisional application No. 61/411,042, filed on Nov. 8, 2010.

(51) Int. Cl. A61F 9/00

(52) U.S. Cl. 604/294

(57) ABSTRACT

A device for delivering an active agent to target tissue at a site that includes a bodily fluid includes a body having a first exterior surface including a first section having a local, discrete recessed area formed in the body for holding the active agent. The body includes a surface flow feature in the form of a canal that is formed in the body and is recessed relative to the exterior surface. The surface flow feature interfaces with the first section and the local recessed area and is configured so as to guide or modify flow of the bodily fluid relative to the body such that fluid communication is provided between the bodily fluid and the local recessed area. The local recessed area is recessed relative to at least a portion of the canal.

### CROSS-REFERENCE TO RELATED APPLICATIONS

The present application claims the benefit of U.S. patent application Ser. No. 61/408,016, filed Oct. 29, 2010; and U.S. patent application No. 61/411,042, filed Nov. 8, 2010, each of which is hereby incorporated by reference in its entirety.

### STATEMENT REGARDING FEDERAL SPONSORSHIP

The U.S. Government has a paid-up license in this invention and the right in limited circumstances to require the patent owner to license others on reasonable terms as provided for by the terms of grant #2 R44 EY013479-04 awarded by the National Institutes of Health.

### TECHNICAL FIELD

The present invention generally pertains to devices for delivery of at least one beneficial active agent to target tissue. More particularly, but not by way of limitation, the present invention has applicability to a device that is situated on the front surface of the eye to deliver an active agent to ocular tissue whereby the active agent is contained in the device and the device has a modified dispensing surface to assist in the release of the active agent to the target tissue.

### BACKGROUND

There are many different types of delivery devices that are used to deliver active agents, such as drugs, to a patient, including but not limited to capsules, implants, etc. One subclass of delivery devices is ocular delivery devices for delivering an active agent to the eye.

With respect to ocular drug delivery devices, approximately 90% of all ophthalmic drug formulations are applied as eye drops. In addition to being difficult for patients to insert accurately, the use of eye drops suffers from two major technical disadvantages, their rapid elimination from the eye and their poor bioavailability to the target tissues. As a result of tear film dilution and elimination and the permeability barriers of the cornea, typically significantly less than five percent of the applied dose of drug reaches the intraocular tissues. Topical ophthalmic pharmaceutical solutions are therefore formulated in high concentrations and require frequent dosing. Non-compliance with treatment, due to required frequency of dosing, lack of detectable symptom relief in immediate association with treatment application, undesirable systemic side effects due to the need for high concentrations of drug and other reasons, is a major clinical disadvantage.

To address these issues the idea of placing a solid device into or near the eye to deliver a beneficial agent for extended periods of time has attracted development work for many years. In general these devices can be characterized as matrix or depot type devices. The matrix device is composed of one material and the beneficial agent is contained throughout this material. A depot device contains the agent in one or more distinct portions of the device. These devices contain a depot of beneficial agent or a depot of material containing the beneficial agent also referred to as a drug depot, drug core, medication depot or simply, a depot. The space in the device's body that contains the depot is referred to by a variety of terms including well, pocket, cache, cavity, reservoir and chamber. U.S. Pat. No. 3,302,646 to Behney discloses a device for bovine ocular drug delivery. The device has a pocket filled with ointment that is held adjacent to the corneal surface and front scleral surface of the eye.

More typically the depot is internal to the device and much of the prior art in these kinds of devices is focused on transporting the drug from the depot to the surface of the device or managing the rate of transport to the dispensing surface.

U.S. Pat. No. 3,416,530 to Ness discloses the use of perforations with capillary action to bring drug to the device's surface from its internal reservoir. U.S. Pat. No. 4,186,184 to Zaffaroni discloses a device with a delivery portal open to that surface of the device that is deemed to be most appropriate for the tissue being targeted.

U.S. Pat. No. 4,973,304 to Graham, et al discloses the use of hydrogel ports to transport drug from the reservoir to the surface of the device.

U.S. Pat. No. 5,902,598 to Chen, et al discloses a device with a diffusion port to transport drug from the reservoir to the surface of the device.

Unfortunately, there are disadvantages and deficiencies associated with each device disclosed in these patents.

### SUMMARY

In one embodiment, a device for delivering an active agent includes a body that has at least one surface for placement proximate to target tissue (e.g., sclera tissue) to which the active agent is delivered. The location of the target tissue also includes a bodily fluid. The active agent is associated and carried by the body in any number of different ways, including but not limited to being disposed within the matrix that forms the body; being disposed in local area, such as in a local recessed area (e.g., a well, pocket or reservoir); being disposed along a surface of the body, etc. Physical features on the surface of the device guide, disrupt or otherwise modify the flow of the bodily fluid relative to the body such that contact is made between the bodily fluid and the active agent for delivering the active agent to target tissue by means of the fluid flow.

When the active agent is disposed within the local recessed area, the active agent is available through an opening thereof to an environment external the device.

In one application, the device is used in an ocular environment. In such an embodiment, the fluid is in the form of ocular fluid (e.g., tears) and one surface of the body is placed in contact with or proximate the target tissue which is in the form of ocular tissues, such as the sclera or other region of the eye. The physical features on the surface of the device guide or otherwise modify the flow of ocular fluid towards, across and/or away from the local area that includes the active agent.

### BRIEF DESCRIPTION OF THE DRAWING FIGURES

FIGS. 1-16 are views of various devices for delivering an active agent to target tissue according to different embodiments of the present invention including an active agent and in some instances, illustrating an exemplary bodily fluid flow pattern showing bodily fluid contacting the active agent.

### DETAILED DESCRIPTION OF CERTAIN EMBODIMENTS

The present application discloses a number of devices for delivering an active agent, which can be in the form of a drug(s) and/or a therapeutic agent, and/or other beneficial agent, to target tissue. The devices are intended for placement in a patient proximate target tissue to which the active agent is delivered. It will be appreciated that the devices can be placed in any number of different locations within the body and therefore, the target tissue can be different tissue found throughout the patient's body.

In one embodiment of the present invention, the device is in the form of an ophthalmic (ocular) delivery device which is part of a topical ophthalmic delivery system and the ophthalmic delivery device is designed to physically direct tear flow at one or more dispensing surfaces of the delivery device for dispensing the active agent (drug and/or therapeutic agent, etc.). As described in detail below, the dispensing surface can be in the form of an exposed surface of the delivery device when the active agent is contained within a polymeric matrix that defines the body of the drug delivery device and/or the dispensing surface can be an exposed surface of an active agent that is disposed within a local recessed space (e.g., a well, cache, compartment, chamber, reservoir, pocket, etc.) that is formed in the body of the delivery device. In this manner, the active agent can be released in a controlled manner to the target tissue.

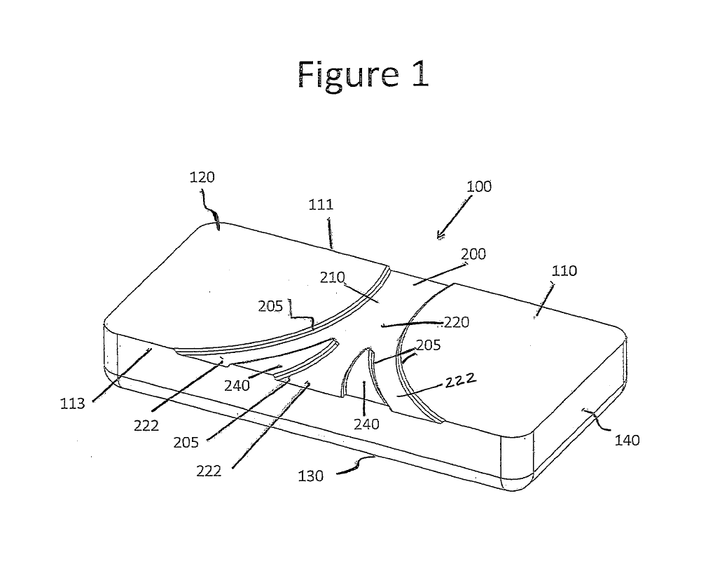

FIGS. 1-3 illustrate a device 100, according to one embodiment, for delivering an active agent. As previously mentioned, the delivery device 100 can be used in any number of different applications relative to the patient's body for treatment of different target tissue sites. While, the device 100 is described herein as being a “drug” delivery device, it will be understood that the device 100, as well as the other devices disclosed herein and shown in the various figures, is not limited to the delivery of a pharmaceutical drug but instead can be used to deliver an active (therapeutic) agent that is not technically classified as being a pharmaceutical drug.

More specifically, the device 100, as well as the other devices disclosed herein and shown in the various figures, is constructed to deliver an active agent to target tissue. The expression “agent” as used herein broadly includes any compound, composition of matter, or mixture thereof that can be delivered from the device to produce a beneficial and useful result. For the purposes of this invention the term medication, medicinal agent, therapeutic agent, beneficial agent or drug can be taken as synonymous.

The devices described in this invention contain an active agent effective in obtaining a desired local or systemic physiological or pharmacological effect. The following classes of active agents can be incorporated into the devices of the present invention.

Suitable drugs or active agents that can be utilized with the present delivery devices include, by way of example only, but are not limited to: (A) Anti-infectives: such as antibiotics, including tetracycline, chlortetracycline, bacitracin, neomycin, polymyxin B, gramicidin, oxytetracycline, chloramphenicol, and erythromycin; sulfonamides, including sulfacetamide, sulfamethizole, sulfisoxazole; quinolones, including ofloxacin, norfloxacin, ciprofloxacin, sporfloxacin; aminoglycosides, including amikacin, tobramycin, gentamicin; cephalosporins; combinations of antibiotics; antivirals, including idoxuridine, trifluridine, vidarabine cidofovir, foscamet sodium, ganciclovir sodium and acyclovir; antifungals such as amphotericin B, nystatin, flucytosine, fluconazole, natamycin, miconazole and ketoconazole; and other anti-infectives including nitrofurazone and sodium propionate; (B) Antiallergenics: such as antzoline, methapyriline, chlorpheniramine, pyrilamine and prophenpyridamine, emedastine, ketorolac, levocabastin, lodoxamide, loteprednol, naphazoline/antazoline, naphazoline/pheniramine, olopatadine and cromolyn sodium; (C) Anti-inflammatories: such as hydrocortisone, hydrocortisone acetate, dexamethasone, dexamethasone 21-phosphate, fluocinolone, medrysone, prednisolone, prednisolone 21-phosphate, prednisolone acetate, fluorometholone, fluorometholone acetate, meddrysone, loteprednol etabonate, rimexolone; (D) Nonsteroidal anti-inflammatories: such as flurbiprofen, suprofen, diclofenac, indomethacin, ketoprofen, and ketorolac; (E) Decongestants: such as phenylephrine, naphazoline, oxymetazoline, and tetrahydrazoline; (F) Miotics and anticholinesterases: such as pilocarpine, eserine talicylate, carbachol, diisopropyl fluorophosphate, phospholine iodide, and demecarium bromide; and (G) Mydriatics: such as atropine sulfate, cyclopentolate; homatropine, scopolamine, tropicamide, eucatropine, and hydroxyamphetamine.

Furthermore, the following active agents are also useful in the present devices: (A) Antiglaucoma agents: such as adrenergics, including epinephrine and dipivefrin, epinephryl borate; β-adrenergic blocking agents, including levobunolol, betaxolol, metipranolol, timolol, carteolol; a-adrenergic agonists, including apraclonidine, clonidine, brimonidine; parasympathomimetics, including pilocarpine, carbachol; cholinesterase inhibitors, including isoflurophate, demecarium bromide, echothiephate iodide; carbonic anhydrase inhibitors, including dichlorophenamide acetazolamide, methazolamide, dorzolamide, brinzolamide, dichlorphenamide; prostaglandins, including latanoprost, travatan, bimatoprost; diconosoids and combinations of the above, such as a β-adrenergic blocking agent with a carbonic anhydrase inhibitor; and (B) Anticataract drugs: such as aldose reductase inhibitors including tolerestat, statol, sorbinil; antioxidants, including ascorbic acid, vitamin E; nutritional supplements, including glutathione and zinc.

Yet another group of active agents is in the form of lubricants: such as glycerin, propylene glycol, polyglycerins and select water soluble polymers, such as the cellulosics, polyethylene oxides, polyethylene glycols and biopolymers such as hyaluronic acid and chitosan.

In addition to the above agents, other agents suitable for treating, benefiting, managing, or diagnosing ocular conditions may be utilized and administered using the sustained release drug delivery devices of the current invention.

In one exemplary application, the drug delivery device 100 is a topical ophthalmic drug delivery device 100 according to one embodiment; however, it will be understood that the device 100 is not limited to only being used in ophthalmic applications but instead can be used in other applications to treat other areas of the body. The drug delivery device 100 is defined by a body 110 that has prescribed dimensions that allow placement in the eye in this particular exemplary application. The body 110 is defined by a first surface or face 120 and an opposite second surface or face 130. A thickness of the body 110 is defined as a distance between the first surface 120 and the second surface 130. The body 110 includes a peripheral edge 140 which in the illustrated embodiment is shown as being a side wall.

It will also be appreciated that the shape of the illustrated body 110 is merely exemplary and the body 110 can have other shapes. The body 110 is formed such that it has a degree of flexibility to allow placement of the body 110 at the target location where the target tissue is located. The body 110 can thus have material characteristics that allow the body 110 to at least generally or substantially adopt the shape of the target tissue to which the body 110 is applied. For example, when used in ophthalmic applications, the body 110 can adopt to the shape of the eye as discussed in more detail below.

It will also be appreciated that either the first or second surface 120, 130 can be placed against target tissue, with the opposite surface thus facing away from the target tissue. In an ophthalmic application, either the first or second surface 120, 130 can be placed against the tissue of the eye as described in more detail below.

It will be understood that while the body 110 has symmetry about a central axis, the device 100 is not limited to having such a characteristic and instead, the body 110 can have an asymmetric construction.

The present Applicant's own previous work describes various ophthalmic drug delivery systems. For example, U.S. patent application Ser. Nos. 10/569,743 and 11/359,156, each of which is hereby incorporated by reference in its entirety. It will therefore be understood that the device 100 as well as the other devices described herein and illustrated in the accompanying figures can be formed of materials that are disclosed in these application and can have structure characteristics that are disclosed in the above applications.

In accordance with the present invention, one or more surfaces of the body 110 includes at least one surface flow feature 200 and/or 240 to guide or otherwise modify the flow of a fluid that comes into contact with the body 110 to assist in delivery of the active agent to the target tissue. In FIG. 1, the surface flow feature 200 is formed in the first surface 120 which can be the surface which faces away from the target tissue.

As shown in FIG. 1, the surface flow feature 200 can be in the form of a locally recessed area that has prescribed characteristics, such as shape and dimensions and contour, which guide or otherwise modify the flow of fluid that comes into contact with the body 110 in order to assist and optimize delivery of the active agent to the target tissue. For example and as described in detail below, the surface flow feature 200 can be constructed to modify the flow of fluid towards the dispensing surface, or away from the dispensing surface, or towards or away from a specific local area defined on the dispensing surface.

The surface flow feature 200 is in the form of a surface feature or modification that defines a fluid flow path along the body of the device for fluid to flow in as the fluid flows along/across the body of the device. The surface flow feature 200 at least partially contains the fluid. The surface flow feature 200 can be in the form of a recessed open canal, groove or other depression having a shape and dimensions that are suitable for guiding or modifying the flow of fluid. The surface flow feature 200 can also be understood to be in the form of a ramp, canal, sluice, valley, half-pipe, cascades, falls, etc., narrowing, broadening, turning, twisting, etc. or in a combination of such forms. In other words, the surface flow feature 200 is in the form of an at least partially open conduit through which fluid can flow and move from one location to another location. While the surface flow feature 200 is discussed herein as being a recessed canal for ease of discussion and uniformity, it will be understood that it is within the scope of the present invention that the surface flow feature 200 can be in the form of any number of different shaped recessed structures. As shown, the surface flow feature 200 is not limited to being of a linear construction but instead, the surface flow feature 200 can have one or more curved sections and can be of a branched construction as shown.

Thus, the surface flow feature 200 can be open along one or more peripheral edges of the body 110. For example, the surface flow feature 200 can be open along two peripheral edges of the body 110. It is also within the scope of the present invention that the surface flow feature 200 is not open along any of the peripheral edges as illustrated and described herein.

In addition, the surface flow feature 200 can be at more than one location along one peripheral edge of the body 110.

It will also be appreciated that the depth of the canal can vary depending upon the particular application and upon characteristics of the body 110, such as thickness of the body 110, etc. In addition, the surface flow feature 200 (canal) can be defined by varying depths in that one area of the surface flow feature 200 can have a first depth (relative to the surface of the body 110), while another area can have a second depth (relative to the surface of the body 110) that is different than the first depth.

As shown in FIG. 1, the surface flow feature 240 can be in the form of a locally elevated area that has prescribed characteristics, such as shape and dimensions and contour, which guide, disrupt or otherwise modify the flow of fluid that comes into contact with the body 110 in order to assist and optimize delivery of the active agent to the target tissue. For example and as described in detail below, the surface flow feature 240 can be constructed to modify the flow of fluid towards the dispensing surface, or away from the dispensing surface, or towards or away from a specific local area defined on the dispensing surface.

The surface flow feature 240 is in the form of a surface feature or modification that alters a fluid flow path along the body of the device or along the canal 200. The surface flow feature 240 can be in the form of a wedge, mound or other elevated shape having dimensions that are suitable for guiding, disrupting or otherwise modifying the flow of fluid. The surface flow feature 240 can also be understood to be in the form of a ramp, bump, column, any other elevated feature that divides, deflects or otherwise disrupts and alters the flow of the ocular fluid over the surface, or in a combination of such forms.

In some embodiments an elevated surface feature may reside within or on a recessed surface flow feature and conversely, a recessed surface flow feature may reside within or on an elevated surface flow feature. Furthermore, an embodiment could have any combinations of these.

However, as illustrated in the accompanying drawings, the surface flow feature can be of a non-branched type which is devoid of surface flow feature 240. In such embodiment, the surface flow feature 200 acts on the flow of the bodily fluid.

Referring specifically to FIG. 1, the surface flow feature (canal) 200 is of a branched construction in that the canal includes a first canal section 210 that is open along a first edge 111 of the body 110 and a second canal section 220 that is formed of a plurality of individual branched canal sections 222 that are in fluid communication with the first canal section 210. The branches are formed by the elevated wedges (raised sections) 240. Each of the branched canal sections 222 is open along a second edge 113 of the body 110. The second edge 113 is opposite the first edge 111. In the illustrated embodiment, there are three (3) branched canal sections 222.

It will be understood that the cross-sectional shape of the surface flow feature (canal) 200 can vary and be selected depending upon the particular application. For example, in one embodiment, the canal 200 can be defined by two opposing walls 210 that are at least generally perpendicular to a canal floor 220. Alternatively, the canal 200 can be defined by two opposing walls 205 that are formed at an angle relative to the canal floor 220. In this embodiment, the walls 205 thus resemble beveled walls as shown in FIG. 1. The angling of the walls 205 creates a beveled canal and can be configured to alter the flow dynamics of fluid traveling into, out of and within the canal. For example, the beveled walls 205 assist fluid flow across such walls to allow fluid to flow more easily into and from the canal 200.

In addition, it will be understood that the individual branched canal sections 222 can have different characteristics relative to one another and in particular, the width and/or lengths and/shapes of the branched canal sections 222 can be different. For example and as shown in FIG. 1, one branched canal section 222 has a greater length; one canal section 222 has a greater width and the canal sections 222 have different shapes.

FIG. 1 shows the device 100 in which the active agent is contained within the body 110 (e.g., dispersed throughout a polymeric matrix that forms the body 110) and is dispensed through one or more surfaces thereof (e.g., surface 120).

When the device 100 is for placement in the eye (i.e., an ocular application), the canal 200 is for guiding and/or modifying the flow of ocular fluids, such as tears. One of the more easily recognized ocular fluids is tears which are necessary for the normal lubrication of the eye and to wash away particles and foreign bodies. As is understood in the art, most of the tears in one's eye are produced by the main lacrimal gland, which sits above and slightly temporal, or away from or opposite the nose from the center of the eyeball. Tear production starts in the lacrimal gland and there are multiple smaller secretory glands located along both the upper and lower lids and in the conjunctiva covering the surface of the eyeball that provide oil and mucous to the tear film. Although the blink mechanism rebuilds a thin film over the exposed portion of the eyeball, at any one inter-blink period, this film represents only a small fraction of the total tear volume in the eye. Conventional tear flow follows a general pathway from the lacrimal gland (source), down over the surface of the eye, and at the same time across the eye towards the nose, where drain holes (puncta) are located. Thus, as the eye lids come together, the lids make a slight conjoined motion towards the nose, thereby pushing the tears in the direction of the drain holes. It will be understood that the foregoing description describing the fluid mechanism of tear flow is not limiting of the present invention but rather describes an accepted mechanism of how tears flow within the eye.

Thus, the surface flow feature 200 acts to direct and guide tears along prescribed flow paths to assist and optimize the release of the active agent that is contained in the body 110.

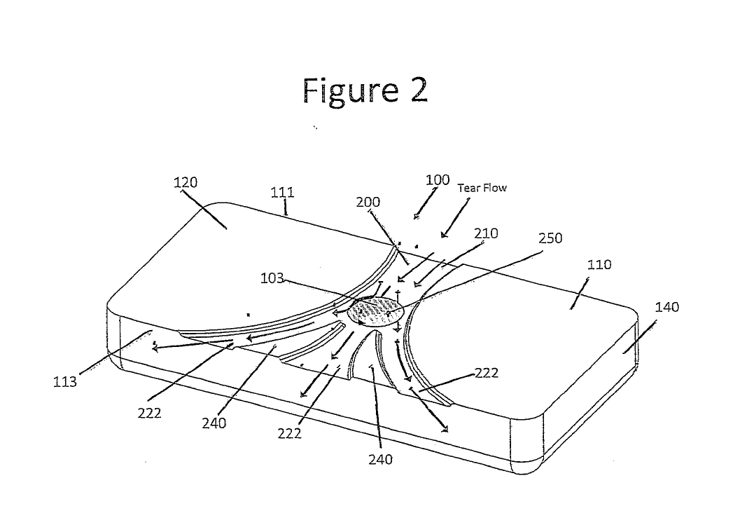

FIG. 2 shows a device 101 according to a different embodiment which is similar to the device 100; however, the device 101 contains an additional feature of a local recessed area (space) 250, such as a pocket, reservoir, chamber, compartment, well, groove, etc. The local recessed area 250 can have any number of different shapes and dimensions that would provide space. It is located and formed such that it is in fluid communication with the surface flow feature 200 but is recessed relative thereto. More specifically, the local recessed area 250 receives or holds the active agent, which is identified in FIG. 2 as 103. As a result, the structural characteristics and positions of the surface flow features 200 and 240 are selected to guide or otherwise modify the flow of ocular fluid towards, across or into, and away from local recessed area 250.

The active agent 103 can take any number of different forms and can have any number of different shapes and have different dimensions. It will be appreciated that the form and dimensions of the active agent 103 depend at least in part on the active agent itself and the specific application. For example, the active agent 103 can have the following forms: a solid tablet, a polymeric matrix containing the active agent, a solid structure containing a liquid active agent in a discrete well, a gel structure, a liquid active agent encapsulated in a structure, a liquid contained underneath a membrane, a polymeric matrix containing the active agent underneath a membrane, or have any other form so long as the active agent 103 can be in communication with the local area 250 and dispersed in a controlled manner.

In FIG. 2, the active agent 103 is located within the surface flow feature (canal) 200 and in particular, the active agent 103 is located proximate the interface between the first canal section 210 and the individual branched canal sections 222. This arrangement allows fluid (e.g., ocular fluid) that flows within the first canal section 210 to come into contact with the active agent 103 and then flow in a direction away from the local area 250 that contains the active agent 103.

As with other embodiments, while not depicted, it will be appreciated that there can be multiple, spaced apart local areas 250 within each surface flow feature 200. These areas can hold the same or different types of active agents.

It will also be appreciated that the direction of flow in the surface flow feature 200 can be either from edge 111 to edge 113 or vice versa. In other words, the fluid can flow in a direction in which the fluid flows into the first canal section 210 and subsequently into the second canal section 220 or from the second canal section 220 to the first canal section 210.

It will also be understood that fluid can enter the surface flow feature (canal) 200 at any number of different locations along its length and therefore, fluid is not limited to initially flowing only into the open end of the canal 200 along the edge 111 but also can flow into the canal 200 at intermediate locations between the ends of the canal 200.

During application, the device 100 is preferably placed at the target location (e.g., within the eye) in such an orientation that optimizes the interaction between the fluid (e.g., ocular fluid) and the surface flow features 200 and 240 to achieve the objectives described herein. In other words, the device 100 is preferably oriented such that the orientation of the surface flow features 200 and 240 are complementary to the flow direction and flow characteristics of the fluid. This results in the fluid naturally flowing within the surface flow feature 200 and coming into contact with the active agent 103. The interaction between the fluid and the active agent 103 serves as a mechanism for effectively dispensing the active agent to the eye and into contact with the target tissue, in this case, ocular tissue. By effectively guiding (directing) the natural fluid that is present at the target location, the natural flow pattern(s) of the fluid is utilized for dispensing the active agent to the target tissue.

The natural fluid thus acts as a carrier for the active agent and directs the active agent across the eye to allow more dispersed and effective delivery of the active agent.

The curved directional arrows showing ocular fluid flow into and out of the local recessed area 250 in FIG. 2 and some subsequent Figs. are merely exemplary and in many circumstances ocular fluid flow may only flow along the external surface of the active agent 103 and does not significantly enter the local recessed area 250.

In the embodiment, the active agent 103 is retained on the body 110 (e.g., within the recessed area) using conventional techniques, including but not limited to use of biocompatible adhesives, physical features in the recessed area 250, physical features in the containment forms of active agent 103, etc.

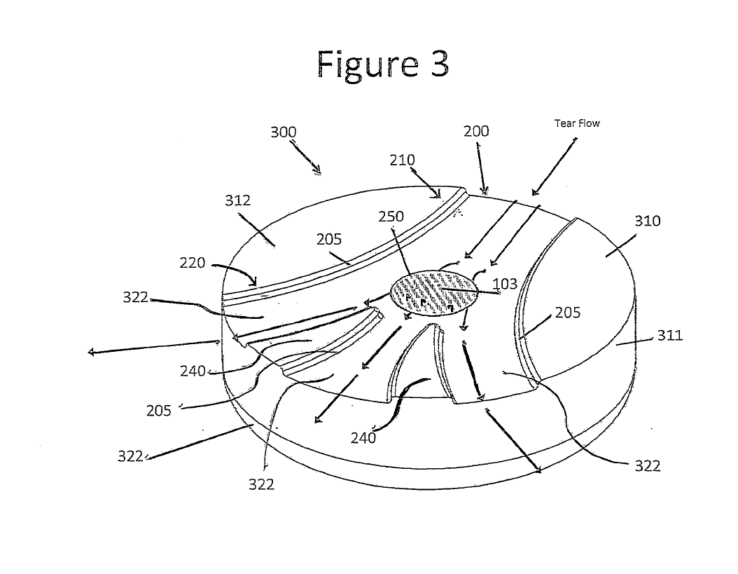

FIG. 3 shows a device 300 that is similar to device 100 except for the shape of a body 310 thereof. More specifically, the device 300 has a circular shaped body 310 and thus resembles a disk or wafer or the like. As with the device 100, the device 300 includes one or more surface flow features 200 and 240 formed along one or more surfaces thereof. In the illustrated embodiment, one surface flow feature 200 is formed along a first surface or face 312. Similar to the surface flow feature 200 shown in FIG. 2, the surface flow feature 200 formed in device 300 is in the form of a multi-branched canal. More specifically, the surface flow feature 200 is in the form of a canal that includes a first canal section 210 that is open along a peripheral edge (circumferential edge) 311 of the body 310 at one location and a second canal section 220 that is formed of a plurality of individual branched canal sections 322 that are in fluid communication with the first canal section 310. Each of the branched canal sections 322 is open along different locations of the peripheral edge 311 of the body 310. In the illustrated embodiment, there are three (3) branched canal sections 322.

It will be understood that all of the structural variations and design details with respect to the surface flow features 200 and 240 described with reference to FIGS. 1-2, apply equally to the surface flow features 200 and 240 formed in any of the other delivery devices described in the present disclosure and illustrated in the accompanying figures, including the device 300. In addition, the device 300, as illustrated, includes the locally recessed area 250 for holding and containing the active agent 103. As with the previous embodiment, the active agent 103 is positioned (and thus the locally recessed area 250 is thus formed) at a location which is in fluid communication with the surface flow feature 200 so as to allow fluid (e.g., ocular fluid) to come into contact with the active agent 103 as a result of the flow path of the fluid being guided or otherwise modified by the surface flow feature 200.

As with FIG. 2, FIG. 3 shows arrows in one direction from the first canal section 210 to the second canal section 220, it will be understood that the flow can be in an opposite direction in that the fluid can flow from the second canal section 220 to the first canal section 210.

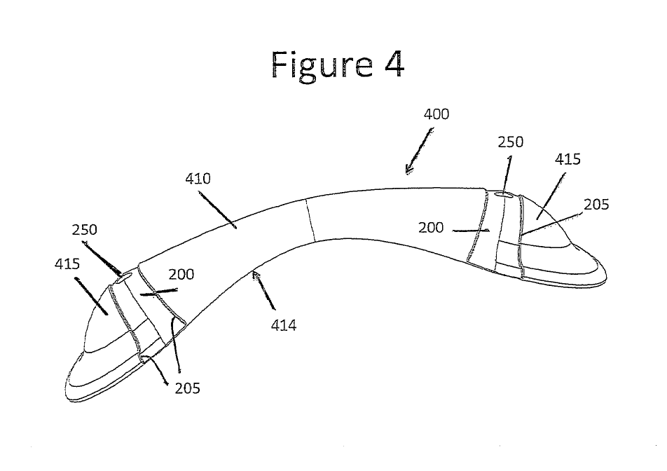

FIG. 4 shows another device 400 for delivering active agent to the target tissue. FIGS. 4 through 20 show many versions of device 400. The device 400 is particularly designed and suited for ocular drug delivery applications and in particular, is constructed for insertion into and wear in the eye by placing it on the inferior or superior anterior sclera (white) of the human eye or in treatment of primates and quadrupeds. As with the previous embodiment, it will be understood and will become more apparent below that the device 400 is merely one exemplary embodiment of the present invention and in no way is to be construed as limiting the scope of the present invention.

The device 400 includes a body 410 that has an edge apex contour 412 which is the amount and positioning of rounding of the device edge and is typically defined as a radius profile swept around a perimeter of the device 400. The device 400 has a base curve (generally identified at 414) which is defined as the primary radius in each meridian i.e. vertical and horizontal, and is the surface of the device 400 that is in general contact with the sclera (the posterior surface of the device). In the case where the values in each meridian are the same, the base curve 414 is defined as a spherical base curve. In the case where the values in each meridian are different, the posterior surface is defined as a toric posterior surface. In any case, the base curve feature refers to the specific curvatures of the posterior surface that are chosen to both fit the device to the eyeball and take into account the interaction of the eyelid with the device, thereby, as is understood in the contact lens design art, balancing the maintenance of proper position with the necessary movement for a non-implanted device to remain biocompatible in the ocular environment. The device 400 also has an edge lift which is a sectional geometry width around the perimeter adjacent to and following the edge apex contour 412 where the base curve 414 is flatter (increased). The edge lift is defined by the incremental radius increase and by a width. The edge lift feature is also understood in the contact lens design art as a specific feature to enhance comfort and allow movement of the device over the ocular tissue without causing irritation or inflammation.

A front curve(s) 418 is defined as the secondary device radius in each meridian i.e. vertical and horizontal (axes defined along the body 410). The front curves generate the surface that is in contact with the lid (the front surface of the device). In the case where the values in each meridian are the same, the front curve 418 is defined as a spherical. In the case where the values in each meridian are different, the front surface of the device 400 is defined as a toric front surface. In one preferred embodiment, the present device 400 disclosed herein, the front curves 418 are defined as toric. The device 400 can also include splines which are geometric entities created by polynomial equations, which define smooth blended contour surfaces bridging from one defined shape or cross-section to another. A lenticular is a manipulation of the thickness of the edge of the device 400 at the front curve geometry adjacent to the edge apex contour on the eyelid side of the device 400. A lenticular can be a positive or a negative curve and typically has a reversed radius direction to the primary front curve radius geometry and the lenticular follows the profile of the edge apex contour 412, thus providing a reduced thickness cross-section profile around the perimeter of the device 400.

The body 410 of the device 400 is constructed and configured to fit the contours of the white part (sclera) of the eyeball itself, while paying tribute to the effects of the eyelids on the position, stability, movement and comfort of the device 400. Although remaining in place, the device 400 also must retain a slight movement with eyelid movement and a slight lag behind movement of the eyeball. This is to permit tear film circulation around the lens to help prevent redness, irritation, adherence to the tissue and build-up of mucus and other surface deposits on the anterior or posterior surfaces. The interaction with the lid is also determined by the design, and, as with a contact lens, will affect the position, stability, movement and comfort of the device 400. Proper interaction of the device 400 with the eyelid also allows flow of the tear film around the device 400, which helps keep it clean of mucous build-up that tends to occur with foreign bodies that are simply trapped in the conjunctival cul-de-sac.

It will be understood that the device 400 of this invention can be worn over the sclera superior to the cornea or inferior to the cornea. It will therefore be appreciated that the delivery devices described herein for ocular applications can be positioned in either of these two locations.

As described in detail in Applicant's previous patent application publications, the device 400 can include one or more lobes 415. In this embodiment, the device 400 generally takes the form of a “dumbbell” with a relatively thin central section and two opposing lobe sections 415 formed at ends of the device. The dumbbell shape of the device 400 redistributes the mass away from the center towards the ends of the device 400, and leads to desired positioning on the sclera under the lid and greater stability on the eye while maintaining volume. The lobes 415 also provide area of increased mass (thickness) that can accommodate the active agent and in particular, each lobe 415 can include one or more local recessed areas 250 for containing the active agent. For example and as illustrated, a local recessed area 250, such as a well, is formed in the lobe 415. In FIG. 4, there are two recessed areas 250, one in each lobe 415, for holding the active agent.

Unlike the earlier embodiments, the surface flow feature 200 shown in FIG. 4 is not of a branched construction in that the surface flow feature 200 more resembles a linear canal. The surface flow feature 200 extends across the width of the device 400 from one edge to the opposite edge. The surface flow feature 200 extends across the lobe 415 (e.g., across an apex thereof) and as in the other embodiments, the surface flow feature 200 is designed to guide or modify the flow of natural fluids (e.g., tears) that surround the device 400. As previously mentioned, the flow direction of the fluid will depend on the location and orientation of the device 400; however, the fluid can flow in either direction within the surface flow feature 200.

As in the other embodiment, the surface flow feature 200 is defined by opposing walls 205 that can be beveled or straight.

It will also be appreciated that in any of the embodiments described herein, the width and length of the local recessed area relative to the width and length of the surface flow feature 200 can vary. The width of the canal is the distance between the two opposing side walls, while the length of the canal is measured from one end to the opposite end of the canal. In particular, the width of the local recessed area (and thus the active agent) can be at least 50% of the width of the surface flow feature or can be at least 70%; or can be at least 90%. In addition, in some applications, the width of the local recessed area can be less than 50% of the width of the surface flow feature. In another embodiment, the width of the local recessed area and active agent can be greater than the width of the canal. With respect to the length of the canal, the active agent preferably is located along less than the entire length of the canal and can represent only a fraction (e.g., less than 25%, less than 10%, etc.) of the overall area of the canal. However, in other designs the percentage can be higher.

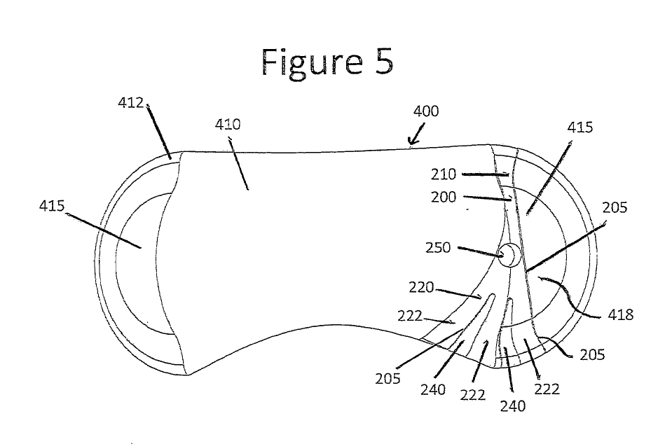

FIG. 5 shows a device 401 that is very similar to the device 400 with the main exception being that the device 401 only includes a single surface flow feature 200 formed on one of the lobes 415. The single surface flow feature 200 is of a branched type similar to the one shown in FIG. 2. It will be appreciated that each of the lobes 415 can include a surface flow feature 200 that contains an active agent either in communication with local recessed area 250. As in all of the embodiments described herein, the depth of the local recessed area 250 depends upon a number of factors including the dimensions of the body of the device, the characteristics, including dimensions, of the active agent, the amount of active agent to dispense, etc.

In the embodiment of FIG. 5, the branched canal sections 222 terminate at or proximate one edge. It is understood that the branched canal sections 222 can terminate at a point not at or proximate the edge of the device. In such a construction, the end of the branched canal section 222 can include a means to facilitate flow of fluid out of or into the branched canal section 222. For example, the end of the branched canal section 222 can include another surface flow feature, a beveled surface (ramp) that serves to cause fluid to flow into or out of the branched channel section 222. As with the other embodiments, there can be two or more branched canal sections 222 when the surface flow feature 200 has a branched construction. In this embodiment, the branches are facilitated by surface flow features 240.

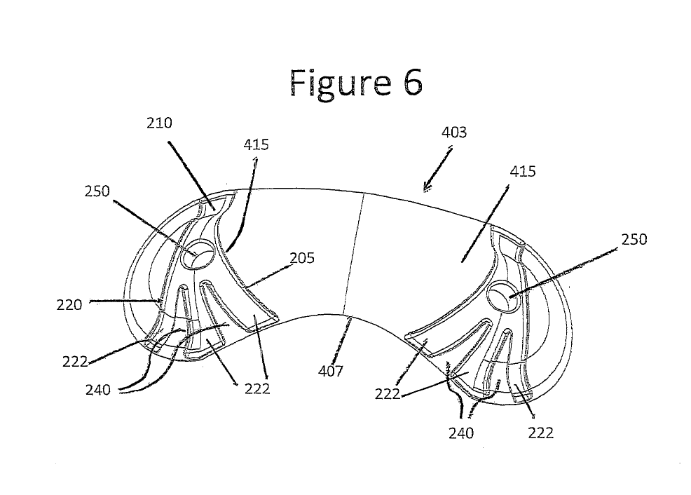

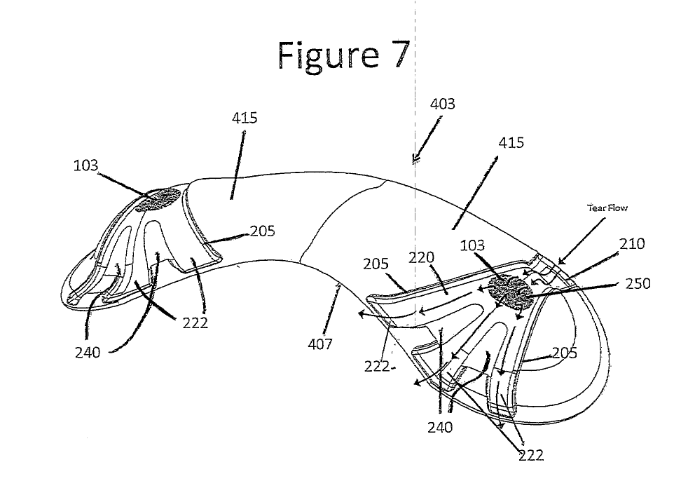

FIG. 6 shows a device 403 that is similar to the other devices 400, 401 and is defined by body 410 and lobes 415. In this embodiment, each lobe 415 includes a surface flow feature 200 in the form of a branched canal and in particular, in the illustrated embodiment, the surface flow feature 200 has three branches (branched canal sections 222). Similar to FIGS. 4-5, fluid is intended to flow along the surface flow feature 200 and it will be understood that the direction of flow can vary depending upon the particular application. For example and as shown in FIG. 7, when edge 407 is positioned proximate and in facing relation to the cornea, the fluid can flow in a general direction from the first canal section 210 to the second canal section 220. However, it is within the scope of the present invention that the flow can be in an opposite direction depending upon the orientation of the device and other considerations.

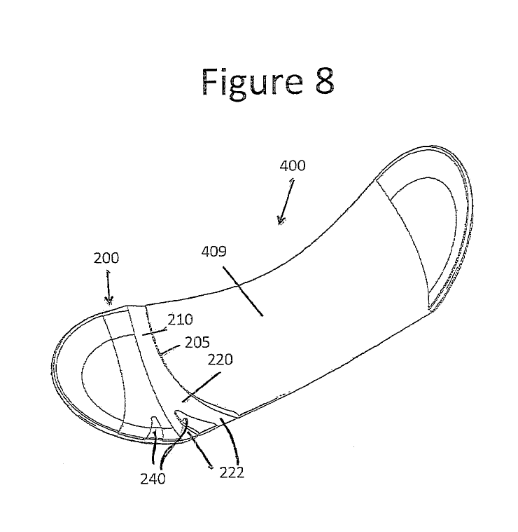

FIG. 8 shows a posterior (underside) surface 409 of the device 400 and in this embodiment, one or more surface flow features 200 are formed along the posterior surface 409.

It will be appreciated that as discussed hereinbefore, the active agent can be dispersed throughout a polymeric matrix that forms the device 400 or it can be located in a local recessed area, such as area 250. As with the surface flow feature(s) 200 formed on the anterior surface of various devices, the formation of the surface flow feature 200 on the posterior surface serves to guide/modify the flow of fluid (e.g., ocular fluid) in such a way that facilities the delivery of the active agent to the target tissue. In other words, the surface flow feature 200 associated with any of the devices described herein serves as a surface flow conduit in which fluid flows into contact with the active agent that is associated with the body of the device.

It will be understood that in any of the embodiments described herein, surface flow features 200 and 240 can be formed on the posterior surface as well as also being formed on the opposite anterior surface of the delivery device.

It will also be understood that in any of the embodiments described herein, a surface flow feature 200 can be formed on the posterior or anterior surface with the feature terminating prior to reaching the peripheral edge of the device, and can utilize a ramp, bevel or useful feature to permit the fluid to more easily flow into or out of the surface flow feature 200.

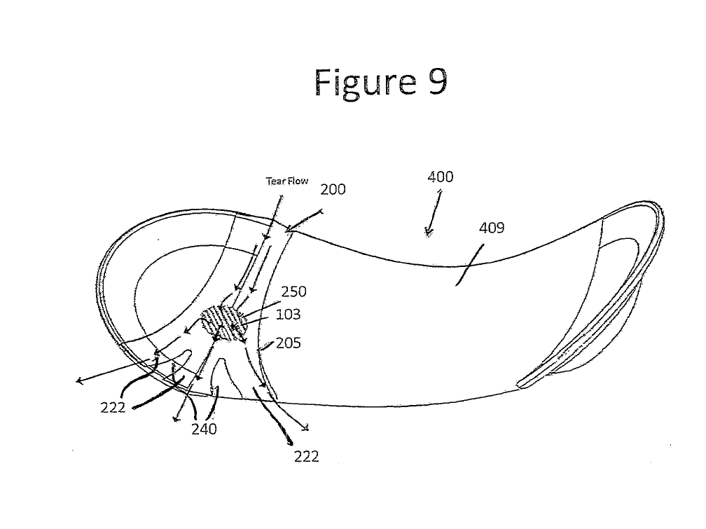

One exemplary fluid flow pattern is shown in FIG. 9. However, as in the other embodiments, it will be appreciated that an opposite flow pattern is equally possible. FIG. 9 shows the active agent 103 being disposed within a local recessed area 250 (e.g., a pocket).

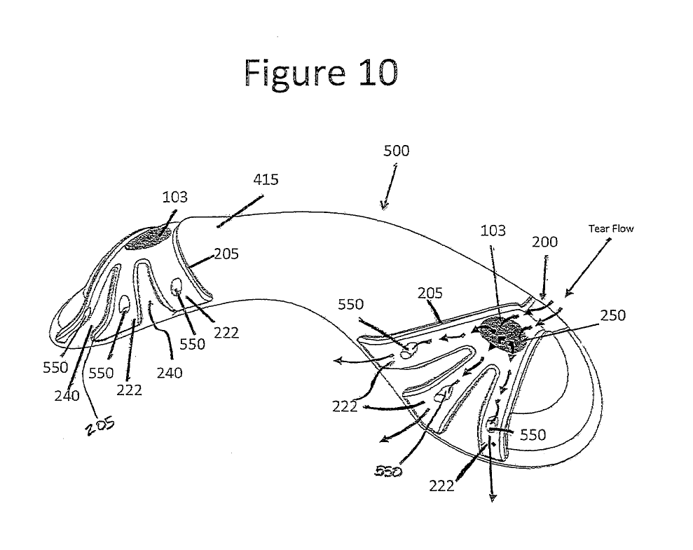

FIG. 10 shows another embodiment that is similar to the previous embodiments, including the embodiment of FIG. 7. In the embodiment of FIG. 10, a device 500 is illustrated. The features of the device 500 that are common to the other embodiments are numbered alike. In contrast to the other embodiments, a body 510 of the device 500 includes a plurality of through openings or passageways or passages 550. The through openings 550 are in the form of openings that pass through the body 510 of the device 500. For example, one or more through openings 550 can be formed in fluid communication with the surface flow feature 200 to provide another or additional means for delivering the active agent to the target tissue (e.g., can be formed directly within the surface flow feature).

In the illustrated embodiment, each of the branched canal sections 222 includes at least one through opening 550. The through openings 550 can be formed anywhere within the branched canal section 222 since this represents a location typically downstream of the source of the active agent 103.

The through openings 550 provide a secondary flow path for the fluid in that some of the fluid can flow within the surface flow feature 200 beyond the through opening 550, while some fluid flows down into the through opening whence it is guided into proximity with the target tissue. The through openings 550 thus provide another means for delivering active agent from the source of the active agent to the target tissue.

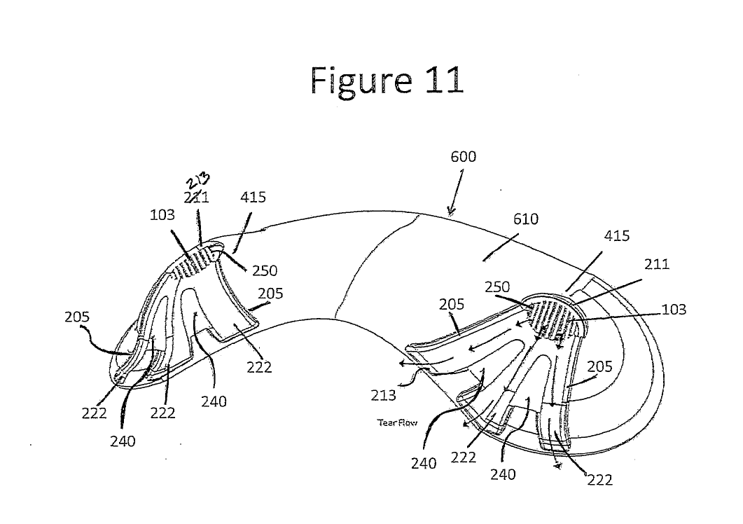

Now referring to FIG. 11, another device 600 for delivering an active agent is illustrated. The device 600 is similar to the other devices described herein. In this embodiment, each surface flow feature 200 (e.g., canal) has a closed end in that the surface flow feature is not open along at least two sections of the edge of the device 600 as in the previous embodiments. In this embodiment, the surface flow feature 200 has a closed first end 211 and an open end 213.

In the illustrated embodiment, the local recessed area (e.g., pocket or reservoir) 250 is located at or proximate to the closed first end 211 of the surface flow feature 200. FIG. 11 shows the surface flow feature 200 having a branched canal construction and as a result, the illustrated surface flow feature 200 is formed of a plurality of branched canal sections 222 that extend toward and terminate at or proximate one peripheral edge of a body 610 of the device 600 so as to facilitate the flow of active agent from the source 103 to the target tissue over which the device 600 is positioned proximately. The branched canal sections 222 are located on one side of the source of active agent 103 and therefore, the flow of tears, assisted by the pumping action of the eyelids and slight movement of the device with blinking, will distribute the active agent released from the device towards the target tissue.

The surface flow feature 200 shown in FIG. 11 can have any of the characteristics described herein with respect to the surface flow feature 200 of other embodiments described and illustrated herein.

The directional arrows showing tear flow are merely exemplary and due to the closed end 211 of this design, the fluid (tears) flows in a direction opposite to and away from the closed end 211 toward a peripheral edge of the body 610 where the active agent carried by the fluid is dispensed to the target tissue.

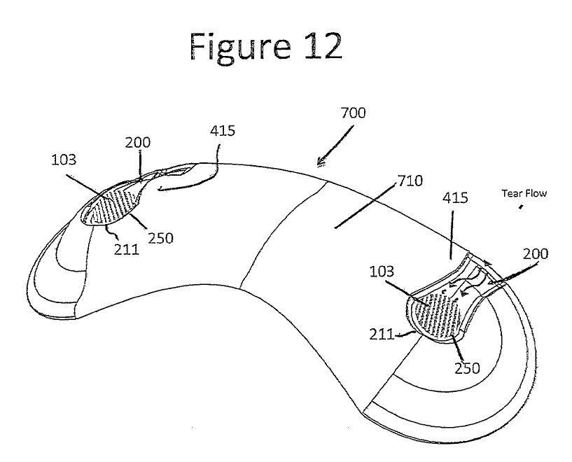

FIG. 12 shows a device 700 that is very similar to the device 600 of FIG. 11 with the exception that the surface flow feature 200 is not in the form of a branched canal construction but rather, the surface flow feature 200 has a more linear construction similar to the one shown in FIG. 4. As with the previous embodiment, the surface flow feature 200 has a closed end 211 that is located at or proximate to the active agent 103 which is shown in the figure as being disposed within the local recessed area 250 (e.g., a pocket).

In FIG. 12, the direction arrows indicate one possible flow pattern for fluid, such as tears, when the device 700 is positioned in the eye such that the surface flow feature 200 extends in a direction radially outwardly from the cornea of the eye and as a result, the normal tear flow pattern (described in detail hereinbefore) results in tears entering the surface flow canal 200 and be guided towards the active agent 103. As in the other embodiments, the tears come into contact with the active agent and the continued flow of the tears facilitates the dispensing of the active agent 103 from its source in the body 710 to the target tissue (i.e., ocular tissue).

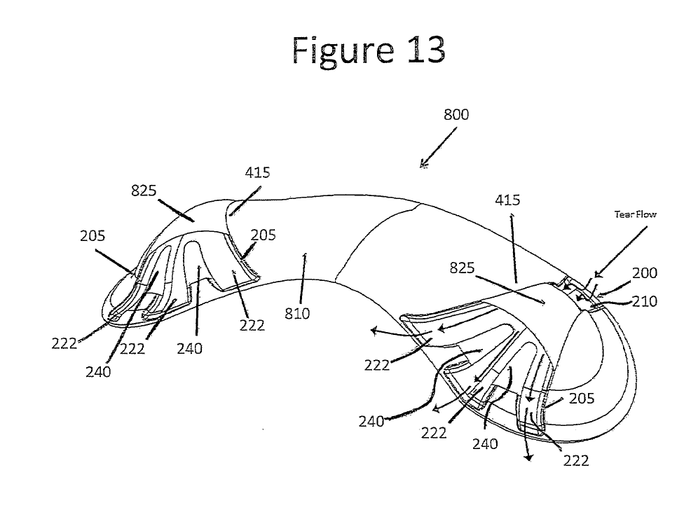

FIG. 13 discloses yet another embodiment of the present invention in which a device 800 for delivering the active agent 103 is illustrated. The device 800 is similar to the device 500 of FIG. 10 and includes a pair of surface flow features 200 that are formed across and at least partially within the lobes 415 of the body 810 of the device 800.

Each surface flow feature 200 is of a branched canal type and is defined by a first canal section 210 that is open along one peripheral edge of the body 810 and a second canal section that is defined by a plurality of branched canal sections 222. The surface flow feature 200 is defined by walls that can include edges 205 that can be straight or beveled. The branched canal sections 222 are open along or proximate to another peripheral edge of the body 810.

While the surface flow features 200 shown in FIG. 13 are of the branched canal type, it will be appreciated that the surface flow features 200 can be of a non-branched type and be more linear in nature as shown in previous figures.

In accordance with this embodiment, a cover 825 is formed across a portion or section of the surface flow feature 200. The cover 825 is preferably located above the active agent 103 so as to further contain the active agent 103 and further influence the dispensing of the active agent 103 by means of the directed fluid, such as ocular fluid, that travels within the surface flow feature 200. It also serves to further prevent direct contact between ocular tissue and the local recessed area 250 (see FIG. 10) and its potentially high concentration of the active agent 103 (see FIG. 10).

In addition, the directional arrows show an exemplary flow pattern for fluid and are not limiting of the present invention. As mentioned herein, the direction and other characteristics of the flow pattern are dependent on certain factors such as the orientation of the device 800.

In effect, a tunnel-like environment is produced by disposing the cover 825 over the surface flow feature 200 (e.g. canal) and this assists in guiding the fluid into contact with the active agent and away from the active agent towards the target tissue. Although not depicted, in some embodiments the cover may be further supported by columns between the cover 825 and the surface flow feature 200.

In one embodiment, the cover 825 is formed of a different material compared to the rest of the body 810 and can have different material characteristics. For example, the cover 825 can be formed of an erodible material such that it erodes over a prescribed period of time. The erosion time frame can be coordinated with the kinetics of the release of the active agent.

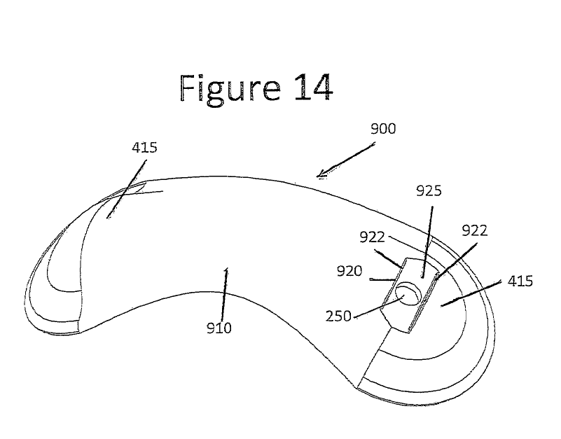

FIG. 14 shows another device 900 for delivering an active agent. The device 900 is similar to other embodiments and includes a pair of lobes 415. In this embodiment, one of the lobes 415 includes a surface flow feature 920 that is recessed relative to the exposed surface of the body 910 and is defined by a planar surface 925 (floor) and a pair of opposing sides 922. The sides 922 can be perpendicular or formed at an angle relative to the floor 925. The surface flow feature 920 does not extend to a peripheral edge of the body but instead is generally confined to being located within the lobe 415. The ends of the surface flow feature 920 are open and allow fluid flow to flow both into and out of the surface flow feature 920.

In this embodiment, the local recessed area 250 is further recessed from the exterior surface (here the anterior surface) of the body 910 and thus the active agent is further recessed.

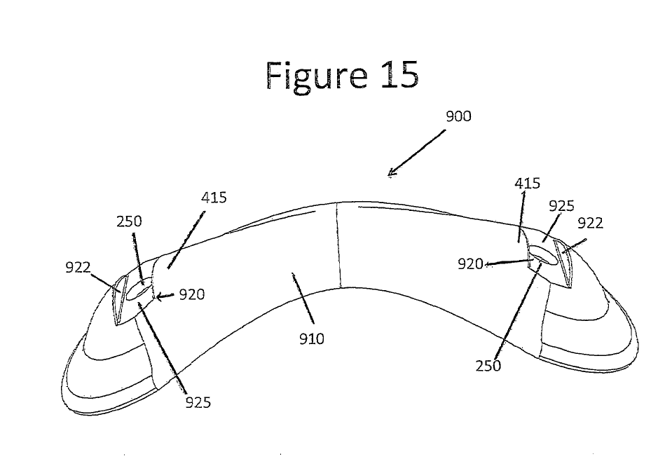

FIG. 15 shows device 900 with the inclusion of a second surface flow feature 920 in the other lobe 415.

As with the other embodiments, the surface flow feature 920 is constructed so as to increase the efficiency of the dispensing of the active agent to the target tissue, and takes advantage of the repeated pumping action of the eyelids to mix the tear fluid over the active agent 103 as the tear fluid slowly flows over the device.

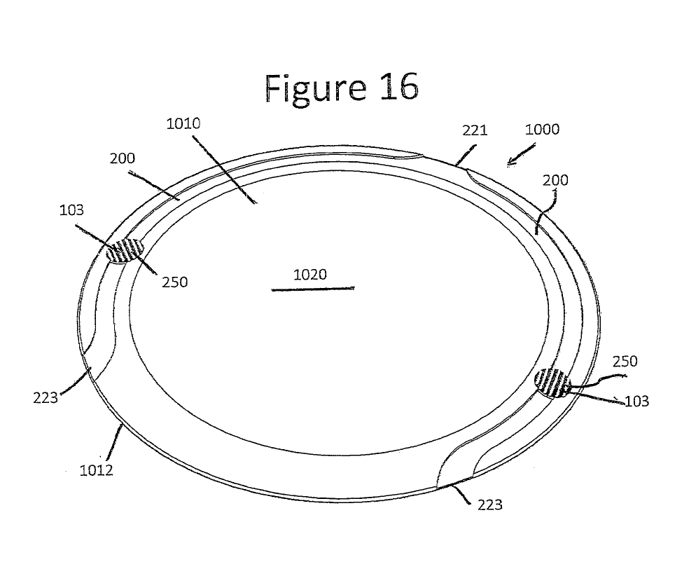

FIG. 16 shows another device 1000 for delivering the active agent 103. In this embodiment, the device 1000 is in the form of a contact lens or the like in that it is formed of a body 1010 that includes an optics region or zone 1020. As shown, the optics region 1020 is centrally located so that when the device 1000 is worn in the eye, the optics region 1020 lies over the cornea. As in the previous embodiments, the device 1000 includes a surface flow feature 200 that is formed in the body 1010 at locations outside of the optics region 1020. As in the previous embodiments, the surface flow feature 200 facilitates contact between natural bodily fluids, in this case ocular fluids (tears) and the active agent 103 so as to facilitate and increase the efficiency of the dispensing of the active agent 103.

As in the previous embodiments, the surface flow feature 200 can take the form of a canal that is formed along a peripheral area of the body 1010 outside of the optics region 1020. In the illustrated embodiment, the surface flow feature 200 is in the form of a canal structure that includes two canal sections that share a common opening 221 that is formed at the peripheral edge of the body 1010. The two canal sections extend in opposite directions along the peripheral edge of the body 1010 and each terminates in an opening 223 at a location along the peripheral edge. The two canal sections are thus arcuate (curved) in nature.

As in the previous embodiments, the canal sections are in fluid communication with the active agent 103 to allow fluid (tears) to contact the active agent and aid in the dispensing and delivery of the active agent 103 to the target tissue. While the illustrated embodiment shows the active agent 103 in a local recessed area 250 (pocket) that is intersected by the canal, it will be understood that the active agent can be carried by the body in any of the other ways mentioned herein, including being disposed in a matrix

It will be appreciated that instead of sharing a common opening 221, each canal section can have a separate opening. In this construction, each end of the canal opens to peripheral edge at two different locations.

In addition, the device 1000 can be constructed to only have one canal and thus one local, discrete area that contains the active agent 103.

The device 1000 functions in the same or similar way as the other devices described herein in terms of delivery of the active agent 103 to the target tissue; the only difference being that it is constructed to be worn in the eye and includes optics region 1020.

In various embodiments, the surface flow feature is in some way an open ended structure in that at least one end thereof is open to allow fluid to pass therethrough in either a direction toward the body of the device or in a direction away from the body of the device towards the exterior of the device. This is in contrast to a conduit that is continuous and closed ended.

When the active agent is disposed in a discrete recessed area (pocket or reservoir), it will be appreciated that the active agent can fill the entire recessed area or can fill less than the entire recessed area and can even overfill the recessed area in that the active agent can be disposed above the opening into the recessed area (i.e., a portion of the active agent can extend partially into the surface flow feature (be above the surrounding floor of the surface flow feature)).

In accordance with one aspect of the present invention, a device for dispensing active agent can consist of a body forming a carrier (body) and drug containing space (drug depot) with one or more dispensing surfaces incorporated onto or into specific surfaces of the device. The drug depot can consist of a three-dimensional space in or on the device containing one or more drugs or drug containing media. The devices of the present invention can be utilized for controlled ophthalmic drug delivery of substances to be distributed into the tear film for greater dispersal to the ocular tissues. Its design technology takes advantage of the physical forces created by blinking and eye movement and facilitates continuous exchange of tear fluid proximate the drug depot's dispensing surface. Its design can also include physical features that reduce or eliminate direct contact between the dispensing surfaces and adjacent tissue. The device configuration is useful for release of drugs such as a prostaglandin analog that might otherwise cause localized irritation, hyperemia, or hyperpigmentation. The device is also useful for a number of conditions including glaucoma, dry eye, infection, ocular surface disorders, and post-surgical healing. The device is particularly useful for releasing glaucoma medications directly into the tear film, thereby supplying drug both via the trans-corneal route into the anterior segment, and via a trans-conjunctival route with broad circumlimbal distribution outside the globe, proximal but external to the root of the iris, for penetration around the entire globe, to the targeted ciliary body and/or episcleral region surrounding the trabecular meshwork. Glaucoma medications are more effective when distributed efficiently to the entire anterior segment tissues that are the target of treatment.

Such delivery of drug is in contrast to concentrating the drug release directly against the tissue from a device with an opening of its drug depot directly over localized areas of tissue as is done with conventional drug delivery devices. Many dry eye medications that act on the ocular surface would also be more effective when distributed to large areas of the ocular surface through the flowing, dynamic tear film. Thus more even distribution to the entire ocular surface where the active agent is needed improves the treatment effect of a given amount of active agent released, while lessening potential toxicity to the tissue immediately adjacent to the opening of the drug depot. The invention when used in an ocular environment works towards more consistent drug release rates, using the tear film acting as an endless sink and active agent dispersion medium, by mixing the tear fluid over the device's drug depot and drawing the drug out of the drug depot, and presenting it via the tear film to large areas of target tissue. This tear fluid route of delivery relies on a concentration gradient between the tear film and the target tissue to help drive the active agent towards the target tissue, rather than on a strictly localized concentration gradient limiting delivery from the drug depot to that localized between the topical device's drug depot surface and the immediately proximal portion of the target tissue.

This delivery alternative can reduce the undesirable side effects of hyperemia, inflammation and hyperpigmentation that can result from concentrated localized delivery proximal to a tissue subject to such side effects, as is seen with repeated topical application of prostaglandin analog drops in glaucoma patients. The device's sustained delivery of drug can eliminate the use of topical eye drops, resulting in improved patient compliance, convenience, and subsequent efficacy. The drug can be incorporated when the device is manufactured, resulting in drug loaded depots with their openings on the anterior, lateral or any surface distal to the surface most proximal to the sclera or bulbar conjunctiva, of the topical ophthalmic drug delivery device.

It will be appreciated that any of the delivery devices disclosed herein, including the ones shown in FIGS. 1-16 can be formed such that instead of the active agent 103 being disposed within the local recessed area 250; the active agent 103 can be disposed along the floor of the surface flow feature 200. In one construction, the active agent 103 can be disposed along the floor in a non-recessed manner relative to the floor itself (but recessed relative to the surface of the body of the device in which the surface flow feature 200 is formed. In other words, the active agent 103 can disposed on a planar or non-planar floor surface of the surface flow feature 200 and constructed in relation to the dimensions of the surface flow feature 200 such that it does not adversely obstruct or impact the flow of fluid within the surface flow feature 200. The active agent 103 can thus be in the form of a film or the like that is disposed along a length of the floor of the surface flow feature 200. The natural fluid (ocular fluid) thus flows over the active agent 103 and the active agent is carried thereby or is otherwise dispensed to the target tissue. In this construction, the active agent can occupy less than the entire surface area of the floor of the surface flow feature 200 and thus be formed in a local, discrete area along the floor. Alternatively, the active agent can be formed in a plurality of local, discrete areas along the floor (e.g., spaced apart areas of the active agent).

What is claimed is:

1. A device for delivering an active agent to target tissue at a site that includes a bodily fluid, with the device comprising:
a body having a first exterior surface including a first section having a local, discrete recessed area formed in the body for holding the active agent; and
a surface flow feature in the form of an open canal that is formed in the body and is recessed relative to the exterior surface, the surface flow feature interfacing with the first section and the local recessed area and being configured so as to guide or modify flow of the bodily fluid relative to the body such that fluid communication is provided between the bodily fluid and the local recessed area;
wherein the local recessed area is recessed relative to at least a portion of the canal.

2. The device of claim 1, wherein the first exterior surface comprises a surface that faces away from the target tissue.

3. The device of claim 1, wherein the first exterior surface comprises a surface that faces towards with the target tissue.

4. The device of claim 1, wherein the local recessed area comprises a well for holding the active agent.

5. The device of claim 1, wherein the canal includes at least one open end.

6. The device of claim 1, wherein the canal is defined by a first section and a second section with the local recessed area being formed within the first section of the canal, the canal being defined by a floor that is recessed relative to the first exterior surface and the local recessed area being recessed relative to the floor.

7. The device of claim 6, wherein the second section comprises a branched canal section defined by at least two branched canal sections, each branched canal section having a first end in fluid communication with the local recessed area and an opposing second open end, wherein the branched canal sections are separated by a divider wall.

8. The device of claim 7, wherein a top surface of the divider wall is at least substantially within a same plane as a top surface of the first exterior surface.

9. The device of claim 6, wherein the first section of the canal is open along a peripheral edge of the body and the second section of the canal is open along the peripheral edge at a different location thereof.

10. The device of claim 1, wherein the body is configured for placement in an eye and the bodily fluid comprises tears.

11. The device of claim 1, wherein the body is formed of a polymeric matrix that includes the active agent.

12. The device of claim 1, wherein a member extends across and over an open end of the local recessed area while maintaining a structure of the canal thereunder so as to further guide flow of the bodily fluid under the member and across the local recessed area.

13. The device of claim 1, wherein the local recessed area is open along a floor of the canal and a width of the opening of the local recessed area is at least 50% of a width of the floor at a location where the local recessed area is formed.

14. The device of claim 1, wherein the local recessed area is open along a floor of the canal and a width of the opening of the local recessed area is at least 75% of a width of the floor at a location where the local recessed area is formed.

15. The device of claim 1, wherein the local recessed area is open along a floor of the canal and a width of the opening of the local recessed area is greater than a width of the floor at a location where the local recessed area is formed.

16. The device of claim 1, wherein the body includes two or more local recessed areas each for holding the active agent and the body also includes two or more canals.

17. The device of claim 1, wherein the body includes at least one through opening formed through a floor of the canal such that it is open along the floor and is open at an opposite end to an opposite second exterior surface of the body, the through opening being configured to permit bodily fluid to pass from the canal to the opposite second exterior surface.

18. The device of claim 1, wherein an opposite second exterior surface of the body includes:
a first section having a local, discrete recessed area formed in the body for holding the active agent; and
a surface flow feature in the form of a canal that is formed in the body and is recessed relative to the second exterior surface, the surface flow feature interfacing with the first section and the local recessed area and being configured so as to guide or modify flow of the bodily fluid relative to the body such that fluid communication is provided between the bodily fluid and the local recessed area;
wherein the local recessed area is recessed relative to at least a portion of the canal.

19. A device for delivering an active agent to scleral tissue in an eye comprising:
a body having an exterior surface and a first section for dispensing the active agent to the scleral tissue, the body being configured to seat against and be retained on the sclera of the eye; and
a surface flow feature in the form of an open canal that is formed in the body, the surface flow feature being configured to guide or modify flow of the bodily fluid relative to the body such that the bodily fluid fluidly communicates with the active agent in the first section, wherein the canal is a non-continuous structure having at least one open end that provides fluid exchange between the canal and an exterior of the device for dispensing active agent to the scleral tissue by providing a flow path across the body of the device over which the bodily fluid flows, the flow path being at least one that: (1) guides the bodily fluid into contact with the active agent and (2) guides the bodily fluid from a source of the active agent in the first section.

20. A device for delivering an active agent to scleral tissue of any eye comprising:
a body having a first exterior surface, the body being shaped and configured to seat against and be retained on the sclera of the eye;
a local recessed area formed in the body, the local recessed area being open along the first exterior surface;
an active agent disposed within the local recessed area;
a surface flow feature in the form of an open canal that is formed in the body and is recessed relative to the exterior surface, the canal intersecting the local recessed area and being configured so as to guide or modify flow of the bodily fluid relative to the body such that the bodily fluid fluidly communicates with the active agent to improve dispensing of the active agent from the body to the scleral tissue;
wherein the local recessed area is recessed relative to at least a portion of the canal.

21. The device of claim 20, wherein the active agent comprises at least one of a drug and a lubricant.

22. The device of claim 20, wherein the bodily fluid comprises tears.

23. The device of claim 20, wherein the canal is positioned and oriented on the body so as to guide the bodily fluid into at least one of contact with the active agent and away from the active agent after the bodily fluid has contacted the active agent.

24. The device of claim 23, wherein the canal both guides the bodily fluid into contact with the active agent and guides the bodily fluid that includes active agent from the local recessed area to the scleral tissue.

25. The device of claim 20, wherein the first exterior surface comprises a surface that faces away from the scleral tissue.

26. The device of claim 20, wherein the first exterior surface comprises a surface that is in contact with the scleral tissue.

27. The device of claim 20, wherein the local recessed area comprises a well for holding the active agent.

28. The device of claim 20, wherein the canal includes at least one open end.

29. The device of claim 20, wherein the canal is defined by a first section and a second section with the local recessed area being formed within the first section of the canal, the canal being defined by a floor that is recessed relative to the first exterior surface and the local recessed area being recessed relative to the floor.

30. The device of claim 29, wherein the second section comprises a branched canal section defined by at least two branched canal sections, each branched canal section having a first end in fluid communication with the local recessed area and an opposing second open end, wherein the branched canal sections are separated by a divider wall.

31. The device of claim 29, wherein the first section of the canal is open along a peripheral edge of the body and the second section of the canal is open along the peripheral edge at a different location thereof.

32. The device of claim 20, wherein a member extends across and over an open end of the local recessed area while maintaining a structure of the canal thereunder so as to further guide flow of the bodily fluid under the member.

33. The device of claim 20, wherein the body includes two or more local recessed areas each for holding the active agent and the body also includes two or more canals.

34. The device of claim 20, wherein the body includes at least one through opening formed through a floor of the canal such that it is open along the floor and is open at an opposite end to an opposite second exterior surface of the body, the through opening being configured to permit bodily fluid to pass from the canal to the opposite second exterior surface.
